(19) United States
(12) Patent Application Publication
(10) Pub. No.: US 2017/0136452 A1
(43) Pub. Date: May 18, 2017

(54) METHOD FOR HIGH THROUGHPUT DISPENSING OF BIOLOGICAL SAMPLES

(71) Applicant: SoluDot LLC, Encinitas, CA (US)

(72) Inventors: Walter D. NILES, La Jolla, CA (US); Jonathan GREY, Encinitas, CA (US); Katherine R. GREY, Encinitas, CA (US); Thomas K. HITTLE, Poway, CA (US)

(73) Assignee: SoluDot LLC, Encinitas, CA (US)

(21) Appl. No.: 15/229,961

(22) Filed: Aug. 5, 2016

Related U.S. Application Data
(60) Provisional application No. 62/255,265, filed on Nov. 13, 2015.

(51) Int. Cl. B01L 3/02; G01N 35/10; C12M 1/36; B41J 2/07; C12M 1/00

(52) U.S. Cl. CPC B01L 3/0268; B41J 2/07; C12M 29/06; C12M 41/48; G01N 35/1009; B01L 2200/143; B01L 2400/0415; B01L 2400/0475

(57) ABSTRACT

A method to obtain high-throughput printing or dispensing of biological samples, especially for use in assay methods employs a continuous flow inkjet printer modified to dispense suitable-sized droplets of biocompatible solutions containing said biological samples.

### CROSS-REFERENCE TO RELATED APPLICATION

This application claims benefit of U.S. provisional application Ser. No. 62/255,265 filed 13 Nov. 2015, the disclosure of which is herein incorporated by reference in its entirety.

### TECHNICAL FIELD

The invention is in the field of biological assays, especially low-volume, high-throughput assays. More particularly, it concerns use of continuous inkjet printing technology to dispense biological samples.

### BACKGROUND ART

Modern biological research requires many diverse assays for specific biological activities in cells or isolated biochemicals for discovery of new biological targets for disease, new medicines directed to those targets, or other chemicals useful in the agrochemical, foodstuffs, cosmetics and other industries. The number of assay types being developed is ever increasing, including detecting pathological genetic mutations, analyzing expression of particular genes, monitoring activation or inhibition of signal transduction pathways, determining enzyme activity, measuring ion channel activity, quantifying levels of metabolites, and other analysis of biological activities and functions. As new assays become developed, they are put to use in discovering the molecular networks involved in normal physiological regulation of cells, control of gene expression, pathogenesis of various diseases, host physiological responses to pathogenic assault, and other knowledge useful in the diagnosis and treatment of disease. Drug development is beginning to require determination of the effects of combinations of chemical compounds on hundreds if not thousands of genes and scores of signal transduction pathways simultaneously to enable design of therapies tailored to the pathogenic mechanisms of a particular disease.

Similar needs arise in molecular biological assays in which it is desired to understand gene expression or the presence of a potentially deleterious mutation. In gene expression analysis, messenger RNA is isolated from cells, reverse transcribed to cDNA, amplified by the polymerase chain reaction (PCR) using non-specific primers, and then hybridized to single-stranded DNA segments encoding particular genes of interest to determine expression levels of those genes. Although these procedures can be routinely performed manually when less than 100 genes activities are assayed, the number of genes of interest may run into the tens of thousands. The need for automation and increased throughput has led to the development of automated thermal PCR cyclers and nucleic acid microarrays. In microarrays, robotic liquid dispensers are used to place nanoliter-volume liquid solutions of DNA segments, which encode genes, on glass or plastic substrates as small pads with length dimensions on the order of tens of microns. In an alternate method to liquid spotting, small, 20-nucleotide fragments of the genes are synthesized photolithographically on the substrate. In genetic mutation analysis, an increasingly common methodology is digital PCR, in which the chromosomal DNA of a patient is isolated and sheared to fragment lengths suitable for amplification by PCR, and then dispensed in a manner such that each test sample contains only a single fragment. Then DNA primers flanking the genetic region of interest containing the sequence that may be mutated are introduced to each sample such that only that region is amplified for probing with a sequence containing the mutation whose presence is desired to be detected.

Therefore, liquid dispensing systems that increase the throughput of each assay on a single platform will benefit the performance of modern biological research and development.

Considerable attention has been devoted to miniaturizing assays while improving the ability of assays to discriminate small changes in biological activity, a process termed “miniaturization.” Miniaturization and parallelization efforts have been advanced by the development of multiwell platforms or microtiter plates. Industry standards have been adopted for the formats and dimensions of these platforms such that the wells are deployed in a consistent manner that enable them to be used across a wide variety of instrumentation. The 8×12 well array, 9 mm well pitch of the 96-well plate has been integrally subdivided to enable 384, 1536, and even 3456 well plates to be standardized to provide platforms for miniaturization efforts. Vendors such as Greiner BioOne, Falcon/Corning, and Nunc make available these standardized platforms. However, successful realization of the true economy of scale requires miniaturization of each assay sample into total volumes within the range of 0.1 to 0.01 milliliters, or less in ways that these assays to provide useful data. This requires maintenance of the accuracy and precision of both the volumes of assay constituents dispensed and their accurate and precise spatial placement within the platform. This further requires utilization of liquid dispensing mechanisms suitable for delivering sub-milliliter to nanoliter volumes necessary for assay construction.

Automated liquid handling is required to achieve the throughput requirements in miniaturized assays. Accuracy and precision of liquid dispensing are most typically characterized for the dispensed volume by the coefficient of variation (CV) calculated by dividing the standard deviation obtained by multiple identical dispenses by the average of the volume dispensed. By far the most common method is automated piston-plunger systems with disposable pipette tips that use positive displacement to effect liquid transfer, such as those offered by Beckman Instruments (Danaher Corp), Thermo Fisher, and others. These systems become unreliable, both inaccurate and imprecise, for volumes <10 μL because of the inherent irreversible thermodynamic nature of liquid detachment from the dispenser tip at slow liquid velocity unless the tip is submerged in well liquid already present. The rated lower CV limit is typically specified as 5% for these instruments. Similar difficulties arise with pin tool liquid transfer to dry wells, where CVs typically range from 30 to 50%. To overcome this limitation, a widely used technology is the solenoid-actuated valve, in which the liquid to be dispensed is maintained at a constant hydrostatic pressure behind the valve, and the valve solenoid is actuated for a few milliseconds to dispense the liquid through an outlet with an orifice diameter of about 100 μm. Instruments using this mechanism are offered by Beckman, BioDot, and others. This mechanism improves volumetric accuracy and precision to a CV in the range of 0.1% at 10 μL, because liquid movements through the orifice subject to the hydrostatic pressure are dominated by ballistic and frictional forces that greatly exceed the entropic forces of liquid-solid adhesion. However, below 1 μL, the CV increases to a range of 5 to 10%, typically because of inherent systemic variability in the hydrostatic pressure used to drive liquid through the orifice, which arises from the flexibility of elastic liquid feed lines to the solenoid valve, and instrument configurations in which the dispenser is moved relative to the target during repositioning necessary for the construction of multiple assays in parallel.

Dispensing assay constituent volumes less than 1 μL, especially in the range of 10 nL or less, which is necessary for miniaturized assay volumes of 10 μL or less, has imposed further difficulties on liquid dispensing systems. The requirement for this volume range arises in assays where relatively small volumes of a chemical compound or biological colloidal concentrate are added to an assay during construction. The need for the small relative volume arises because the compound may be dissolved in a non-aqueous solvent, such as dimethylsulfoxide or benzene, which may exert its own effect in a biological assay. The objective is to dilute the small volume of solvent (e.g., 1 nL) with the relatively much larger volume of aqueous assay diluent (1 μL) to a concentration where biological effects are mitigated. Another need is to enable reconstitution of an aqueous concentrate of a biological material isolated under biochemical conditions that may interfere with the performance of a particular assay into a more favorable environment. This situation arises in the screening of chemical compound libraries for new therapeutics and general molecular biology procedures. To obtain these low volumes, inkjetting technologies such as thermal- or piezo-actuation have been adapted to biological assay construction. Two commercially available piezo-actuated dispensers for miniaturized assay construction are the Microdrop from PE Biosystems and the PicoRAPTR™ from Beckman. A new technique for small-volume dispensing is surface acoustic wave control in which the surface of the liquid to be dispensed is energized to produce a standing stationary wave. Energization is provided by a small acoustic lens, such as a curved piezoelectric ceramic lens brought into contact with the bottom of the container of the liquid. Dispensing of pico- or nano-liter sized drops is actuated by the addition of a high-amplitude transient pulse to the energizing wave, which causes reorganization of standing wave modes into a jet that projects from the liquid surface and coalesces into a drop the volume of which depends on the amplitude of the actuation pulse. Commercial systems from EDC Biosciences and Labcyte are available for this type of dispensing. However, these mechanisms of inkjetting present their own difficulties for accurate and precise dispensing on the microscale. Thermal inkjetting is often rendered unusable simply because of heat denaturation of the biological colloidal material, which not only degrades biological or biochemical activity but also fouls the dispensing orifices. Piezo-actuated dispensing from microcapillaries at net zero imposed hydrostatic pressure, i.e., from a liquid interface at atmospheric pressure suffers when liquid residue from prior dispenses altering both the volume and trajectory of subsequent ejected drops. Acoustic systems are profoundly sensitive to the spatial configuration of the liquid interface of the source material, which is often a small, 2 mm or so, diameter well of a plastic microtiter plate, that can deleteriously affect both the trajectory and volume of the ejected drop. Therefore, automated liquid dispensing for biological and biochemical assays would greatly benefit from the adoption of technologies capable of better volumetric and trajectory control.

Continuous inkjetting has been used commercially in industrial printing for labeling a wide range of products. The operating principle of continuous ink jetting is that the liquid to be printed is transported out of a storage reservoir to a pressure chamber with an opening orifice on the side that faces the target to be printed. Typical orifice diameters range from 20 to 200 μm. To create individual liquid drops in the continuous liquid jet emitted from the orifice, the pressure chamber is attached to a modulation element that vibrates the liquid to create an elastic pressure wave along the surface of emitted jet. The pressure fluctuations in this elastic wave cause the liquid to break apart into individual droplets of uniform volume by the Rayleigh principle a short time at a specified distance after the jet front exits the orifice. An individual electrical charge is imparted to each droplet, with the magnitude of the electrical charge dependent on its desired spatial location on the target at impact, by directing the liquid jet stream, just prior to breakup into individual drops, through a pair of charging electrodes. With the liquid reservoir behind the orifice held at ground potential, and because the liquid has net non-zero ionic strength, and, hence, electrical conductivity, electrostatic induction enables free charge carriers in the liquid to be moved toward or away from the charging electrode pair by varying the polarity and amplitude of said electrode pair. Because the droplet separates from the leading edge of the jet within the electrical field between the charging electrodes, the induced charge separation at the edge remains on the droplet after separation at a magnitude corresponding to the charging electrode voltage difference but with its polarity reversed. An additional electrode located downstream of the point of separation in the space between the charging electrodes may be used detect and measure the charge imparted to each drop such that deviations from the desired charge amplitude may be fed back to correct the charge amplitudes of subsequent droplets. The charged droplets continue on a linear trajectory into a constant electrostatic field within a downstream plate capacitor wherein they are deflected at specific angles from their initial linear trajectory as a function of their charges, such that after they leave the deflection field, they continue to travel along their deflected paths to impact specific spatial locations on the target. Liquid droplets that are not to be directed to the target are programmed to have zero net charge, or a charge of an amplitude enabling them to remain undeflected from the linear direction at which they are emitted from the orifice, such that they enter a collection tube. The collection tube enables recirculation of the unused liquid back to the liquid supply reservoir, hence the term continuous ink jet.

While continuous inkjet printers are commercially available, because of the constrictions on the nature of the droplet formation imposed by the apparatus, such printers have not been considered appropriate for use to dispense biological samples because the nature of the fluids needed for such samples was thought to be incompatible with the requirements of such printers. In particular, as further described below, a parameter called the Ohnesorge number (Oh) which is dependent on the characteristics of the liquid to be dispensed, was required to be at a level not obtainable by solutions with viscosities or other physical characteristics that are suitable for maintaining the integrity of biological samples. Thus, typically, liquids to be dispensed using such printers are adjusted to have viscosity, density and surface tension characteristics that result in a satisfactory value for the Oh number. Such adjustments would lead to inactivation or otherwise harmful effects on biological samples including living cells contained in the liquid.

It has now been found that, by suitable adjustments of the parameters affecting the printing apparatus, liquids suitable for biological assay can be accommodated in this system, despite the failure of these liquids to achieve an Oh number considered necessary for successful uniform sampling.

### DISCLOSURE OF THE INVENTION

The present invention provides a continuous liquid jet printing system and method of dispensing liquids of biological and biochemical utility to permit high throughput dispensing of nanoliter quantities of reagents and other solutions, including cell suspensions, for construction of biological and biochemical assays. These assays employ miniaturized formats suitable for biotechnological experimentation. As noted above, continuous liquid jet printing has not been applied to biological testing because the parameters associated with such printing have not been compatible with liquids of biological utility.

The sources of these incompatibilities include the colloidal nature of many biological and biochemical solutions and suspensions, including proteins, nucleic acids, and cells and the nature of solutions and suspensions required for maintaining the activity and/or viability of biological materials, which were believed to prevent modulation of the liquid jet into individual droplets of uniform volume that can be charged in a manner suitable for trajectory control.

The present invention surprisingly overcomes these limitations by providing continuous liquid jet dispensing systems amenable to the use of biological solutions and suspensions. The formulations compatible with both biological and biochemical materials and continuous liquid jet dispensing requirements are dispensed with accuracy and precision of the individual droplet volumes and of trajectory control. Thus, amounts of biological materials quantitative at the microfluidic nanoliter scale are reliably dispensed and printable on a target stage to enable reliable assay construction.

Thus, in one aspect, the invention is directed to a method for high-throughput dispensing of biocompatible medium containing a biological sample which method comprises applying uniform droplets of said medium to predetermined positions on a target by dispensation by a continuous inkjet printer.

In particular, the invention is directed to a method wherein said applying is by generating droplets of said medium of uniform size by electrostrictive modulation; steering said droplets to predetermined positions on a target by charging the droplets through a pair of electrodes such that the amount of charge on each droplet determines the spatial position of said droplet on the target; synchronizing the charge on each droplet with passage of the droplet between the charging electrodes by correction of the phase of application of the charging voltage; and deflecting the droplets to said predetermined positions by passing said droplets through an electric field for deflection of said droplets, such as a plate capacitor.

Thus, a continuous inkjet system suitable for carrying out the method of the invention comprises a dispensing reservoir for a liquid from which the samples are to be taken, an orifice through which the liquid passes, modulating elements that provide a modulating frequency to vesiculate the liquid into droplets, charging electrodes to provide each droplet with a different charge, and an electric field typically a capacitor, which deflects the droplets to predetermined positions. According to the method of the invention, the parameters associated with these components are maintained and controlled. In some instances, this is done automatically through coupling a continuous inkjet printer to control units which maintain the appropriate values of these parameters.

Thus, in another aspect, the invention is directed to a system for high-throughput dispensing of biocompatible medium containing a biological sample which system comprises a continuous inkjet printer and at least one computerized control unit wherein said control unit adjusts the operating parameters of the continuous inkjet printer to be satisfactory for dispensing said medium.

More particularly, the invention is directed to a system wherein the inkjet printer comprises a dispensing reservoir, an orifice through which said medium is dispensed, a modulating element for vesiculating said liquid into droplets, charging electrodes to provide electrical charge to said droplets, an electric field such as a capacitor for deflection of said droplets, and wherein said control unit(s) adjusts the pressure in the dispensing reservoir, the frequency of the modulation, the voltage difference between the charging electrodes and the voltage difference between the plates of the deflecting electric field.

### BRIEF DESCRIPTION OF THE DRAWINGS

FIG. 1 shows a simplified block drawing of an example embodiment of a continuous liquid dispensing system useful in the present invention.

FIG. 2 shows a schematic view of an example of a control system for continuous liquid dispensing in accordance with the present invention.

FIGS. 3A and 3B show results of dispensing a saline solution compatible with biochemical and biological assay constituents compared to saline containing glycerol used to obtain greater solution viscosity.

FIG. 4 shows accurate spatial placement by continuous liquid dispensing of 10 mM fluorescein in Dulbecco's Phosphate Buffered Saline (DPBS) by the use of automated target positioning according to the method of the invention.

FIG. 5 is a graph of number of drops of 10 mM fluorescein in DPBS dispensed per well vs. fluorescence. A linear relation between the number of drops and intensity of fluorescence is obtained.

FIGS. 6A-6C show images of murine neural stem cells at different times after dispensing to a 6-well tissue culture plate. FIG. 6A shows cells immediately after dispensing;

FIGS. 6B and 6C, respectively, show the cells at 3 days and 6 days. Enough cells are viable such that a confluent culture is attained by 6 days.

### MODES OF CARRYING OUT THE INVENTION

The method and system of the invention utilize an apparatus for continuous inkjet printing that is commercially available. For example, the MD4 printer available from PrintSafe, Poway, Calif., can be used and is described in US2009/0277980. Although the apparatus itself has known features, it has been used only to dispense fluids that are inappropriate for use in biological assays. It was believed that these characteristics of the dispensed fluids were necessary in order to provide successful and error-free printing. As will further be explained below, the invention method permits this type of apparatus to be used to dispense biological samples and includes both a method for sampling biologically compatible fluids and a system for controlling the parameters of the apparatus to permit such a method to be successful.

### Apparatus and System

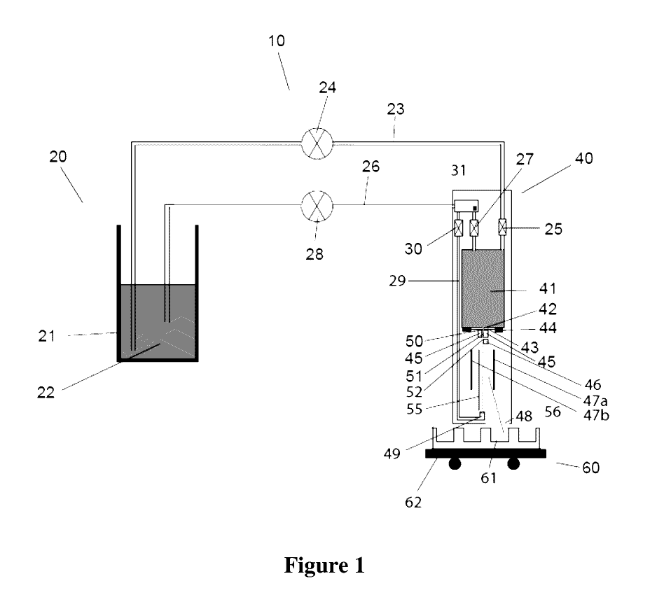

In order better to understand the nature of the method and system of the invention, a description is provided of the apparatus to which the method and system apply. The general features of the type of continuous liquid dispensing apparatus used in the invention method and system are illustrated schematically in FIG. 1 and are not to scale for the sake of clarity.

FIG. 1 shows these features of the continuous liquid dispensing apparatus 10 used in the invention, which comprises an external fluid system 20, a dispense head 40, and a sample stage 60. In the external fluid system 20, an external tank or reservoir 21 contains the liquid to be dispensed 22. Said liquid is conveyed to a dispense head liquid reservoir 41 through a fluid supply tube 23 by means of a fluid supply pump 24 that provides a positive hydrostatic pressure difference to drive liquid into the dispense head liquid reservoir 41. The amount of liquid delivered is controlled by means of a fluid supply valve 25. The hydrostatic pressure of the liquid in said dispense head liquid reservoir is maintained constant by a fluid return tube 26 with a return control valve 27 interposed between the dispense head liquid reservoir 41 and the fluid return or suction pump 28 that conveys liquid back to said external tank. During normal dispensing operations, the hydrostatic pressure of the liquid in said dispense head liquid reservoir is maintained constant by control of said fluid supply and return pumps.

Liquid in the dispense head liquid reservoir 41 is continually ejected out of the reservoir through an orifice 42 that in general has a diameter in the range of 30 to 80 μm, and in some embodiments of 36, 55, or 70 μm drilled through a supporting plate 43 attached to the reservoir. The diameter of said orifice is selected to produce droplets of a desired volume, that may range from 0.1 to 100 nanoliters (nL), and typically 1 nL. The supporting plate is mounted to the dispense head in a manner allowing its replacement with an orifice of the needed diameter. In one embodiment, said liquid 22 in said reservoir 41 is vibrated by means of mechanical coupling to an electrostrictive mechanism such as a piezoelectric transducer 44. Said transducer is driven by a sinusoidal alternating voltage from an oscillator (not shown) at a constant frequency, termed the “modulation frequency”, preferably in the range of 70 to 120 KHz, to induce a longitudinal elastic wave along the surface of the liquid jet 50 emerging from said orifice. This elastic wave causes the emerging liquid jet to vesiculate into individual droplets. It is known from the Rayleigh principle that the time between drops is identical to the period of the modulation. A pair of charging electrodes 45 is located below said orifice, such that the spacing between said electrodes is between 0.5 and 3 mm, and generally 1 to 2 mm. The charging electrodes are positioned such that the liquid jet 50 emitted from said orifice is approximately equidistant from each electrode in the intervening gap space, and, so that the point of separation of the first liquid droplet 51 from the leading edge of said liquid jet 50 is located within the gap.

Electrical charge is induced in the leading edge of liquid jet 50 and first separated liquid droplet 51 by pulsing an electric field between said charging electrodes, such that the magnitude of charge induced in a droplet is can be varied in a stepwise manner by increases or decreases in the voltage difference between the charging electrodes. Each charged droplet 52 descends along a vertical trajectory 55 (shown parallel to but displaced from the actual trajectory in FIG. 1) past a phase detector 46. Said phase detector is driven at the modulation frequency to sample the amplitude of the induced charge on the drop relative to the amplitude of the applied charging electrode voltage pulse. By means known to those skilled in the art (e.g., U.S. Pat. No. 4,435,720), the phase of the charging voltage pulses relative to the modulation (droplet-generating) frequency is adjusted so that the desired amplitude of charge is induced in each droplet selected for dispensing.

Droplets descend along said vertical trajectory to the space between the plates of the deflecting capacitor. In one embodiment, this capacitor comprises a high-voltage plate electrode 47a and a ground plate 47b. Electrode 47a is located on the side of the droplet vertical trajectory toward the opening 48 in the bottom of the dispense head 40 through which droplets dispensed toward the target 61 located on the sample stage 60. The voltage across the plate capacitor is held at a controllable amplitude to deflect charged droplets away from vertical trajectory 55 and toward dispensing trajectory 56, such that the angle of deflection is proportional to the amplitude of the induced charge on each droplet. Uncharged droplets remain undeflected toward the dispensing trajectory and continue along the vertical trajectory to the collecting tube inlet 49 to the gutter fluid tube 29. Said inlet is held at ground potential and the gutter fluid circuit is held at negative hydrostatic pressure by pump 28 when the gutter return valve is open. The undispensed liquid is returned to the external liquid reservoir 21 for recirculation through the fluid circuit via a manifold 31 that allows mixing of the return liquid from the dispense head liquid reservoir with the undispensed liquid from the gutter fluid tube. In an alternate embodiment, a single high-voltage plate 47a is used to deflect the charged droplets toward the target. Said single plate is located on either side of vertical trajectory 55. The entire dispense head is fabricated of an electrically conductive material such that it may be maintained at ground voltage relative to the plate. The plate is held at a constant voltage amplitude with a sign necessary to deflect droplets with net induced charge toward dispensing trajectory 56 and out of the dispensing head through opening 48. Either configuration of deflection electrodes is referred to as a deflection field.

The dispensed droplets are delivered to a sample target 61 mounted on an x-y linear translation stage 62. Said translation stage is used to position a plurality of locations of the target under opening 48 of the dispense head 40 such that controlled patterns of dispensing, either the number of droplets delivered and the spatial location of each droplet, can be delivered to each target location in succession whereby experimental assays can be composed.

From the description above, it is evident that the location on the plate 60 to which a droplet is dispensed is determined by a combination of the x-y position of the plate 60 and the magnitude of the charge on the droplet which controls the angle at which the droplet is deflected.

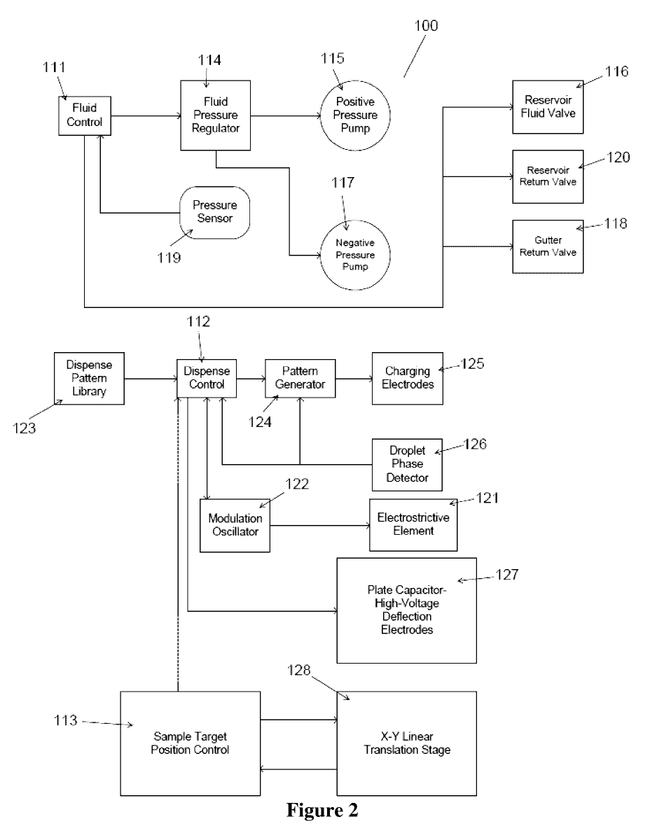

FIG. 2 shows one embodiment of the continuous liquid dispensing system of the invention where relevant parameters are controlled by computers. The computer controls include a host computer running a dispensing control system 100 comprising fluid control 111, dispense control 112, and target positioning 113 subsystems. Fluid control maintains a net positive hydrostatic pressure difference of liquid in the dispense head liquid reservoir at a measured value of about 1000 to 3000 mbar and typically 1400 to 3000 mbar relative to atmosphere by means of fluid pressure regulator 114. In operation, positive pressure pump 115 continuously moves liquid into said dispense head liquid reservoir with reservoir fluid valve 116 open, and negative pressure pump 117 continuously sets the collection tube at a pressure of −10 to −50 mbar relative to atmosphere with gutter return valve 118 open to return fluid to the external tank. The pressure sensor 119 in the return manifold feeds back the measured pressure to fluid control 111 so that opening or closing reservoir return valve 120 enables regulation of an independent flow of liquid out of the dispense head to maintain constant head pressure.

Dispense control 120 also sets the frequency at which the electrostrictive element 121 modulates the liquid jet ejected from the orifice by control of the modulation oscillator 122. The dispense pattern at each dispense actuation is encoded as an entry in a library 123 tagged with the following data: the sequence of droplets in the stream on which to induce a net charge commencing after actuation of the dispense command, and the amplitude of charge to be induced on each of those droplets in the sequence. The pattern generator 124 converts the library entry tag into a temporal pattern of voltage pulses with variable amplitudes proportional to the amount of charge to be induced on each droplet of the sequence delivered to the charging electrodes 125. For example, a dispense actuation sequence pattern of {0, 0, 1, 0, 0, 1, 1, 1} with a charge amplitude set of {0, 0, 5, 0, 0, 4, 3, 2} specifies that the third, sixth, seventh, and eighth drops in the stream after an actuation is commenced are charged with voltage amplitudes of 5, 4, 3, and 2, respectively. To ensure that the dispense pattern is delivered to the charging electrodes in synchrony with the actual temporal distribution of droplets generated by the electrostrictive element, the droplet phase detector 126 measures the phase difference between the time of detection of the droplets and the modulation oscillator clock. This enables the pattern generator to temporally offset the dispense pattern to ensure that the droplets are efficiently charged at the correct time, such that the droplets are charged to amplitudes necessary for the voltage difference between the high-voltage deflection electrodes of the plate capacitor 127 to deflect the charged droplets from the droplet stream to accurate and precise locations on the sample target.

Dispense control 120 may also utilize sample target position control 113 by means of an x-y linear translation stage 128. Dispense actuation may be synchronized with target movement, such that a plurality of dispense actuation droplet sequence and charging amplitude patterns are delivered to the pattern generator in response to the linear translation stage moving either to an absolute position of the target or to a position a relative distance from the prior actuation. This enables dispensed droplets to be delivered to the sample target in 2-dimensional patterns to ensure favorable delivery of assay constituents during construction, such as to favor dissolution of reagents in a diluent or other purposes. In addition, target movements may be effected to allow delivery of different types of patterns to a plurality of sample targets, such as the wells of a microtiter plate, enabling different quantities of assay constituents to be placed in different wells in a controlled way.

### Sample Medium Requirements

A solution composition appropriate for use in biological assays contains water as the principal vehicle or carrier medium. For dispensing according to the invention, it is further desirable to include at least one electrolyte, such as a simple binary salt, e.g., sodium chloride, potassium chloride, or other alkali halide, to provide the solution composition with an ionic strength necessary for charge induction on the liquid droplets.

A primary electrolyte requirement is that the salt be favorable for the performance of the assay, i.e., that it not be an inhibitor of, for example, the catalysis velocities of the various enzymes specific to the biochemical activity desired to be measured in the assay.

A secondary electrolyte requirement is that the salt dissociate sufficiently to provide the desired ionic strength, which, in turn, determines the specific electrical conductivity of the solution. Preferred solution specific electrical conductivity for induction of electric charge on the droplets is at least 1 mS/cm at about 25° C. and more desirable electrical conductivities of 10 mS/cm or more are preferred that may be obtained at a solution concentration, for example, of 0.17 M sodium chloride, or an ionic strength of 0.34 M. In the case of simple binary alkali halide salts, e.g., sodium chloride, dissociation is considered “complete” because the ionic bonds between Na⁺ and Cl⁻ in the undissociated salt are broken by the hydration of each ionic species to the extent that undissociated salt is undetectable. The upper limit for electrolyte composition is determined both by the solubility of the salt and by the tendency of high salt concentrations to affect the colloid stabilities of potential solution components such as peptides, proteins, nucleic acids, polymer carbohydrates, and other biochemicals. Therefore, it is desirable for the final solution composition to have a specific conductivity less than 30 mS/cm, which corresponds to an ionic strength of less than about 0.5 M.

It is further desirable for the solution composition to include buffers to stabilize or keep constant pH of the solution. Acceptable buffers are well known. Some examples of combinations that meet the electrolyte and pH buffer requirements are pre-mixed commercially available compositions, such as, for example, phosphate-buffered saline, Dulbecco's Phosphate Buffered Saline, Hank's Buffered Saline Solution, or other compositions of salts and well-known buffer systems suitable for specific biochemical assays.

In order to permit reliable and precise dispensation of sample droplets, the dispensed liquid must be amendable for the stable breakup of the liquid ejected through the dispensing orifice such that droplets of constant diameter are constantly and uniformly formed along a fixed trajectory. Certain physical properties of the liquid and various force parameters of ejection through an orifice interact to determine the stability of jet formation and its breakup into uniform droplets. These properties and force parameters include:

(i) the inertia of the liquid is characterized by its density ρ, and thus describes the ballistic force that needs to be applied to a unit volume of the liquid to accelerate it from rest within the dispense head reservoir to the terminal velocity of the jet;

(ii) the interfacial surface tension γ describes the force necessary to increase the surface area of the liquid jet as liquid is added to it during ejection; and

(iii) the dynamic viscosity μ describes the internal friction within the liquid that must be overcome as liquid is driven through the orifice and into the forming jet.

Theoretical treatments of drop formation have been used to determine limits of these parameters that ensure drop stability. For example, if the surface tension γ of the liquid relative to its density ρ is too great, then an exceedingly large force will be required to eject the liquid into a jet from an orifice to the extent that the jet may form at all. Conversely, if the ballistic force on the liquid greatly exceeds the frictional retardation of flow, the liquid will spray from the orifice. However, the major objection to the use of water or dilute aqueous solutions or suspensions in continuous ink jetting has been the contention that the surface tension and density are too great relative to the viscosity, such that the jet will protrude too far from the orifice before vesiculation. This will allow the propagation of multiple frequency modes of the elastic wave created by the vibration of the electrostrictive element. Without damping, these higher modes will cause the jet to break up into droplets of different sizes, which is termed “satellite formation.”

In an effort to understand and mitigate satellite formation, the scaling parameter termed the “Ohnesorge number” has been used to evaluate the suitability of liquids for continuous liquid dispensing. In general, Ohnesorge numbers >0.1 are believed necessary to avoid satellite formation. The Ohnesorge number (Oh) is calculated as

$$Oh = \frac{μ}{\sqrt{ρ \cdot γ \cdot d}} \qquad ( 1 )$$

where d is the characteristic length of the jet (orifice diameter), and μ, ρ and γ are viscosity, density and surface tension, as noted above. When comparable units of μ, ρ, γ and d are selected, the units cancel out and the calculated Oh value is a pure number. Thus, Oh expresses the relative balance of forces in droplet formation comparing the frictional damping of liquid flow into the jet to the ballistic force imparted to the liquid and the force necessary to increase the jet's surface area. By the criterion of Eqn. 1, pure water at a temperature of about 25° C. ejected through an orifice of 60 μm would be expected to form satellites. Water at 25° C. has density ρ=997 Kg-m⁻³, interfacial surface tension γ=7.25×10⁻² J-m⁻², and dynamic viscosity μμ=8.9×10⁻⁴ Pa-s. Thus the calculated Oh of the emerging water jet is 0.035, and, hence should be subject to satellite formation.

It is thus standard practice in continuous liquid jet dispensing to add agents to an aqueous composition that increase viscosity and decrease its interfacial surface tension and/or density, in order to increase Oh. In many cases, a single agent is used that performs all three adjustments. For example, polyhydric alcohols, polyhydric ethers derived from these alcohols, hydroxyl- and carboxy-methylated celluloses or other monomeric or polymeric agents that decrease interfacial surface tension and density, and increase viscosity are often specified as desirous in liquid compositions. These components typically exert only partial mitigation of satellite formation, as seen in an example formulation specification for aqueous dispensing solutions containing complex amphiphilic acrylate-aliphatic polymers in combination with aliphatic alcohols (U.S. Pat. No. 8,455,570), in which the interfacial surface tension (γ) is between 0.02 and 0.06 J-m⁻², dynamic viscosity (μ)=1.5 to 3×10⁻³ Pa-s, and a density (ρ) is on the order of 1000 Kg-m⁻³ typical for water. Since addition of various components can only increase Oh to a range of 0.025 to 0.087 for the 60 μm diameter orifice, this is still under the criterion.

One aspect of the invention is to control the apparatus used in the invention to become suitable for dispensing fluids that are acceptable as samples for use in biological assays, which precludes use of components which greatly enhance the viscosity of the medium so as to be incompatible with cell viability or enzyme catalysis, for example. Instead, it has surprisingly been found that the systems and methods of the invention can mitigate satellite formation and dispensing defects by adjustment of the physical parameters of the dispensing apparatus to favor stable ejection of the aqueous liquid jet through the orifice and uniform vesiculation into droplets. These physical parameters are

the pressure of the liquid in the dispensing liquid reservoir of the dispensing head,

the orifice diameter,

the modulation frequency of the electrostrictive element;

the voltage difference of the charging electrodes, and

the voltage difference of the deflecting capacitor.

The theoretical basis of Oh rests on the ratio of two time scales important to jet breakup—the Rayleigh time scale of jet breakup into droplets

t_{R}=\sqrt{ρ·d³/γ} (2)

which is on the order of 30 to 80 μsec for an aqueous solution jetted through an orifice of 40 to 80 μm diameter, respectively. This means that the interfacial surface tension drives the jet to vesiculate at a rate of 10 to 30 KHz. The other time scale reflects the time course over which interfacial surface tension is limited by viscous drag of liquid into a forming droplet

$${t}_{visc} = \frac{μ \cdot d}{γ} \qquad ( 3 )$$

which is <1 μsec for an orifice diameter of 80 μm or less.

This reveals why aqueous jets tend to form satellites—the interfacial surface tension is so strong relative to the frictional forces limiting liquid movement in the jet that thinning dynamics are too fast, because the interfacial surface tension is so much greater relative to the viscous drag that a long jet will be snapped into multiple droplets almost 100 times faster than the speed of propagation of the elastic wave that would drive orderly vesiculation of the jet.

### Controlling the Parameters

The problem of satellite formation by aqueous liquids is solved by this invention by ejecting the liquid through the orifice at low positive pressure, and vibrating the electrostrictive element at a sufficiently high frequency such that the length of the jet continuously protruding through the orifice is short enough so that only one or a few droplets are formed at breakup of the leading surface of the jet.

As noted above, the parameters must be adjusted so as to accommodate the typical characteristics of media that can support biological materials, including living cells. Briefly, these parameters including density, surface tension, and viscosity are in the following ranges:

ρ 900-1200 kg-m⁻³ or 0.9-1.2 g/ml

γ 7-10×10⁻² J-m⁻² or 70-100 erg/cm²

μ 7-15×10⁻⁴ Pa-s or 0.7-1.5 centipoise

As also noted above, the parameters to be adjusted are as follows:

the pressure of the liquid in the dispensing liquid reservoir of the dispensing head,

the orifice diameter,

the modulation frequency of the electrostrictive element;

the voltage difference of the charging electrodes, and

the voltage difference of the deflecting electrodes.

These parameters are interrelated in determining the stability of aqueous droplet generation. Thus, for example, the orifice diameter will determine the necessary pressure of the liquid in the dispensing reservoir as well as the modulation frequency of the electro-restrictive element. Typical values for the voltage of the charging electrodes are in the range of 15-27 V, and of the deflection electrodes of 5 to 7 kV. Under these conditions, typical values for the head pressures and modulation frequencies are shown in Table I below with more detail provided with respect to these parameters in following three paragraphs.

It will be evident that suitable values for any orifice diameter with respect to the remaining parameters can be calculated based on the relationships shown in Table I. Thus, the preferred parameter settings are those that are based on these relationships.

In one embodiment, with a 55 μm diameter orifice, the positive pressure of the dispense head liquid reservoir is regulated to be within the range of approximately 2400 to 2700 mbar such that the leading edge of the liquid jet reaches a length of about 120 μm before each droplet breaks off. In an alternative embodiment with a 36 μm diameter orifice, the dispense head reservoir pressure is held within the range of 3400 to 3700 mbar. In another embodiment with a 42 μm orifice, this pressure range is 2700 to 3200 mbar, and in another embodiment with a 70 μm diameter orifice, the pressure is held at 2300 to 2500 mbar to achieve breakoff of a droplet from the jet of uniform volume.

Using these new typical settings, the jet is modulated by application of the appropriate alternating sinusoidal voltage to the electrostrictive element used to vibrate the orifice and adjusting the frequency to match the selected orifice diameter, i.e., the frequency of this voltage is selected according to the orifice diameter and the viscosity of the liquid to be dispensed. The preferred range of frequencies for a liquid with viscosity in the range of 0.8 to 1.1×10⁻³ Pa-s ejected through a 55 μm diameter orifice is 90 to 95 KHz with a preferred setting of 92,165 Hz. Table I shows frequency ranges and preferred settings for different orifice diameters.

TABLE I
Preferred modulation frequencies for continuous liquid
jet dispensing of aqueous solutions

| | Low freq. | High freq. | | |
|---|---|---|---|---|
| Orifice Dia. (μm) | (KHz) | (KHz) | Preferred (Hz) | mbar |
| 36 | 105 | 110 | 109,097 | 3400-3700 |
| 42 | 100 | 105 | 104,701 | 2700-3200 |
| 55 | 90 | 95 | 92,165 | 2400-2700 |
| 70 | 65 | 70 | 68,423 | 2300-2500 |

Once a modulation frequency is set for vibration of the orifice, and a dispense head reservoir pressure is set such that the leading edge of the liquid jet is located in the top ⅓ of the space between the charging electrodes, the voltage of the modulation frequency is adjusted to achieve continuous vesiculation of the leading edge to a droplet of uniform size such the droplets generated in this way emerge from between the charging electrodes equally spaced in distance. This is achieved by direct visual observation with the aid of a microscope built into the side of the dispense head with its optical axis aligned perpendicular to the trajectory of the emerging droplets, a field of view encompassing both the space between the charging electrodes and about 10 mm of distance below said electrodes, and stroboscopic illumination synchronized with the modulation frequency. The preferred voltage is thus determined empirically and ranges from 15 to 27 V, but a typical value for aqueous solutions containing biochemical and biological materials is 23 V.

Thus, by balancing the diameter orifice with the reservoir pressure, which can be done manually or using the system of the invention, it is possible to control the remaining parameters of the continuous dispensing apparatus so that it successfully and reproducibly provides droplets of suitable, uniform size without satellite formation or spraying for biological testing using media for the tests that are compatible with this purpose.

The voltage difference between the deflection electrodes of the deflection field is set so as to achieve the desired sample pattern essentially free of defects. This is fixed to a range of 5 to 7 KV and preferably at about 6 KV when the opening through the bottom surface of the dispense head is located a vertical distance of 5 to 25 cm (the dispense height) above the target surface on which the liquid is dispensed. The amplitude is adjusted for both the dispense height and the length of dispense pattern delivered to the target surface.

The polarity of the deflection electrodes is set to match that of the charging electrodes. For example, if the charging electrodes have a left-to-right polarity of net positive, the droplets are charged to a negative left-to-right polarity. When these droplets pass through the space between the deflection electrodes held at a left-right net positive polarity, the charged droplets are deflected away from the vertical axis extending from the orifice to the collection tube and toward the dispensing trajectory to the target surface.

According to the invention, all of these parameters can be controlled by manual selection of the various parameters, including modulation frequency of the electrostrictive element, voltage amplitude of the modulation signal, deflection voltage, and pressure of the dispense reservoir, which are input to the computer-based dispensing control system depicted in FIG. 2 through a graphical user interface. These parameters are set according to the desired droplet volume whose range is determined by the selected orifice diameter mounted to the dispense head. And based on the orifice settings, these parameters are adjusted to permit the stable dispensation of biologically compatible fluids. Droplet trajectories are controlled by sequences of voltage amplitude pulses applied to the charging electrodes. These are encoded into a dispense pattern library that also can be selected and modified through the graphical user interface.

Thus, by controlling these parameters, it is possible to dispense a variety of specific media dependent on the assay. For instance, for biological assay constituents such as cells, it is desirable to match the density of the aqueous liquid to the density of the cells. This enables a homogeneous distribution of cells throughout the solution and prevents their collecting in the external liquid reservoir and removal from solution by descent under gravity due to the mismatch in buoyancy. Cell density matching agents include electrolytically neutral sucrose and/or other saccharides, as well as sucrose and other saccharides polymerized to high mass branched polymers with high water solubility that are used in gradient ultracentrifugation to isolate biological cells. Polymerized sucrose is commercially available as a sterile preparation called Ficoll™ available from GE Healthcare. The typical density of 1050 Kg-m⁻³ is typically matched by 10% (w:v) Ficoll™ in the dispense liquid, and the polymer is preferred to sucrose due to its much lower effect on the activity of water, and, hence, osmotic pressure compared to sucrose, so that the cells are not depleted of water. Other desirable constituents for the composition of continuous liquid jet dispensing solutions containing biological cells include solutions formulated as “growth media” containing salts, metabolizeable saccharides, amino acids, hormones, fatty acids, phospholipids, vitamins, proteins, nucleosides, and other nutrients fostering cell growth and survival well-known to those skilled in the art.

The following examples are offered to illustrate but not to limit the invention.

### Example 1

### Comparison of Dispensing of Biocompatible Medium to Dispensing of Standard Medium

The effectiveness of dispensing Dulbecco's Phosphate Buffered Saline (DPBS) (137 mM NaCl, 2.67 mM KCl, 8.10 mM Na₂HPO₄, 1.47 mM KH₂PO₄) with and without 10% vol/vol glycerol was compared. Seven point five grams (7.5 g) fluorescein sodium salt to 100 ml of each solution was used as a marker and each solution was adjusted to pH 7.4 resulting in a final fluorescein concentration of 10 μM.

Each solution was added to the external liquid reservoir of a SampleMaker continuous liquid dispensing system (Inkdustry gmbH, Tauberbischofsheim, Germany) equipped with a 55 μm diameter orifice, and was pumped through the system with a positive pressure of 2600 as measured by the SampleMaker pressure control system. This pressure produced the most stable modulation of droplet generation as determined by observation of the continuous phase control output of the drop phase detector. The electrostrictive element was vibrated with a sinusoidal voltage of 92,165 Hz frequency and 20 V amplitude to obtain droplets of uniform size with minimum satellite formation.

A banner logo 5 mm high and 70 mm long was dispensed onto a graph paper target (with 6.5 mm grid spacing) placed on the top surface of a manually moveable linear translation stage. Each typographical letter character pattern in SampleMaker is encoded as a set of vertical strokes dispensed in succession along the horizontal width of the character in a matrix 7 mm vertical height and 5 mm horizontal width. Each stroke comprises a series of charging electrode pulses of progressively greater amplitude that locate the dispense trajectory of every other droplet along the vertical stroke at each horizontal position. The dispense head bottom surface was located 1 cm above the target. The stage triggered a 24 V pulse output to the SampleMaker to actuate dispensing after a movement of 5 mm.

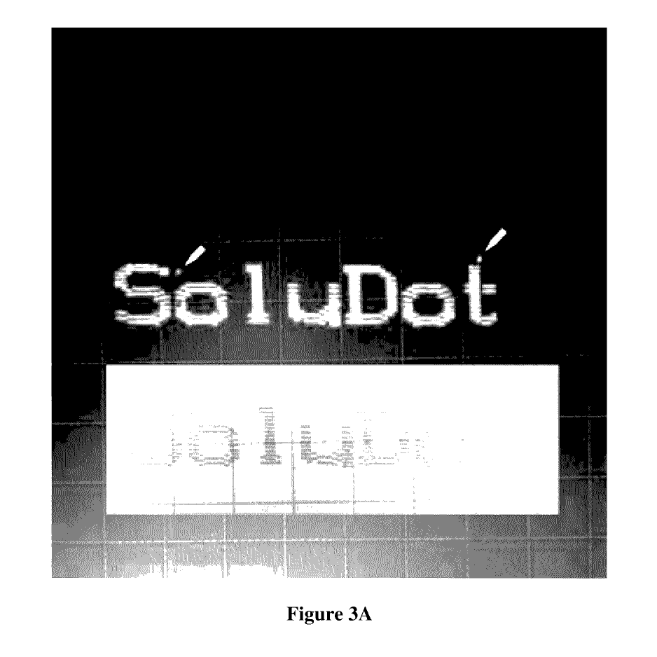

FIG. 3A shows a photograph of the pattern generated by the glycerol-containing sample illuminated under ultraviolet (350 to 400 nm) light.

The lower image of FIG. 3A shows an unprocessed form of the dispensed pattern. Drop dispense defects above the left side of the typographical letter character ‘o’, and above the ‘t’ are marked with white arrowheads in the upper image where the light and dark picture element brightnesses of the lower image are inverted. In addition, there are visible drop displacement defects in the cusp portion of the letter ‘u’.

FIG. 3B shows an inverted image of the same dispensed pattern but obtained without added glycerol. The SampleMaker was washed with ethanol and then DPBS before plumbing with fluorescein in DPBS. The liquid was pumped through the dispense liquid reservoir at a pressure of 2650 mbar, and the voltage amplitude of the modulation frequency (92,165 Hz) was adjusted to 21 V to obtain uniform, equally spaced droplets. The logo dispensed pattern was actuated in the same manner as above, with a horizontal position signal from the manually operated linear translation positioner, and the paper target was displaced vertically between actuations to obtain 3 copies of the pattern on the target.

Referring to FIG. 3B, the same dispensed pattern used in FIG. 3A is repeated 3 times using fluorescein DPBS without glycerol. This reveals fewer errors in placement of dispensed droplets on the target. The droplets dispensed to form the cusp of the character ‘u’ are significantly more in alignment with the desired placement. The most significant error is the visible ‘drop out’ in the one stroke of the bottom of the character ‘D’ in the middle dispensed pattern. Visual observation of the droplet stream emerging from the jet both within the space between the charging electrodes and in the 5 mm below revealed that the droplets were consistent in spacing and uniform in size without formation of satellites.

Therefore, the dispensing liquid formulations used in the method of the present invention in which simple electrolytes are used to create properties favorable to dispensing biological samples not only are surprisingly acceptable, but result in fewer errors that could affect assay composition.

### Example 2

### Automated Accurate Spatial Placement of Aqueous Assay Component Solutions by Continuous Liquid Dispensing

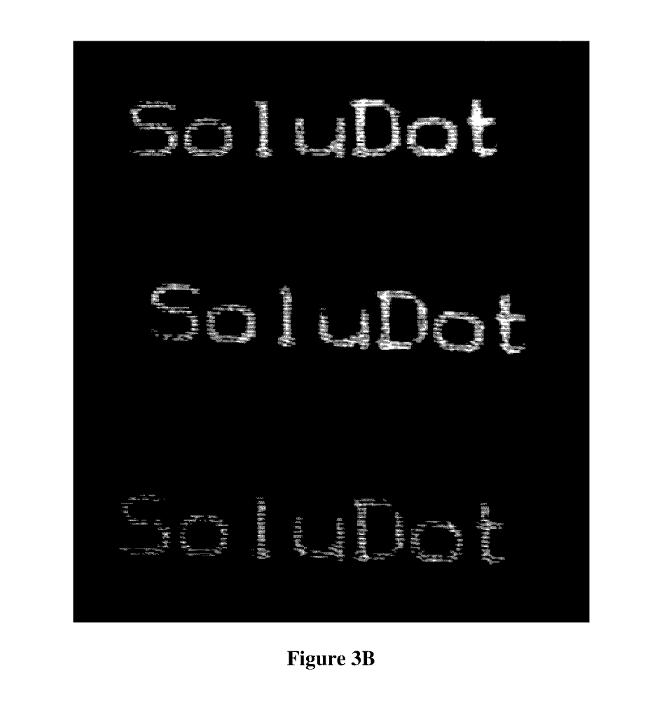

A 10 mM fluorescein-DPBS solution was dispensed in a custom pattern consisting of a single vertical stroke comprising 8 droplets toward a paper target. The target was placed on an automated X-Y planar translation stage (EXCM-30, Festo, Inc., Hauppauge, N.Y.) and the dispense opening of the dispensing head was placed over a spatial location of the target that was referenced with respect to the homing position encoded in the positioning software that was operated on a host computer separate from the dispensing controller host of the SampleMaker. This reference location was used to drive the target under the dispensing head to locations separated by 9 mm spatial displacements in both horizontal and vertical directions. Dispensing of the stroke pattern was actuated manually under SampleMaker control after each displacement step of the motion plan was executed and the positioner automatically came to a stop. The resulting pattern of lines shown in FIG. 4 demonstrates these compositions appropriate for biological assays can be dispensed as accurate samples to targets such as multiwell microtiter plates.

### Example 3

### Automated Dispensing to Multiwell Microtiter Plate

Volumetric accuracy and precision of dispensing was assessed by dispensing 8 droplets of 10 mM fluorescein-DPBS to each well of a 96-well microtiter plate. These wells have an ANSI industry-standard 9 mm distance in X- and Y-directions between the centers of each well. The A1 well of the plate was aligned under the dispensing head opening and used to reference 9 mm traverses of the plate in x- and y-directions under the dispensing head controlled by a motion plan in the Festo. A single droplet of solution was ejected to each well under actuation control of the SampleMaker. For all 96 wells, the dispense pattern was a single picture element, meaning that all dispensed droplets were charged to the same amplitude and delivered to the same spatial location without vertical stroke. In 48 wells, the 8 droplets were delivered with a single actuation. In the remaining 48 wells, the 8 droplets were delivered in 2 actuations of 4 droplets per actuation each. Each well was then diluted with 0.1 mL of dye-free DPBS, and the fluorescence of each well at 530 nm wavelength with illumination at an excitation wavelength of 480 nm was read with a fluorescence plate reader (Envision, Perkin-Elmer, Inc.) that uses a photomultiplier tube (PMT). The average fluorescence in each well for the single actuations of 8 droplets was 522,602±2560 (±standard deviation) absolute PMT counts (CV=standard deviation/average=0.49%) and the average fluorescence for the wells in which 2 actuations of 4 drops each was 522,608±2954 counts (CV=0.57%), which are statistically indistinguishable. These results were consistent for 6 plates dispensed in this way.

Single droplets were also dispensed to each well in 96-well microtiter plates. In this case, a gain of 2.0 was applied to the PMT signal output. The average fluorescence per well was 146,905±7739 for a CV of 5.2%, which is consistent with other nanoliter dispenser methods used for automated assay construction.

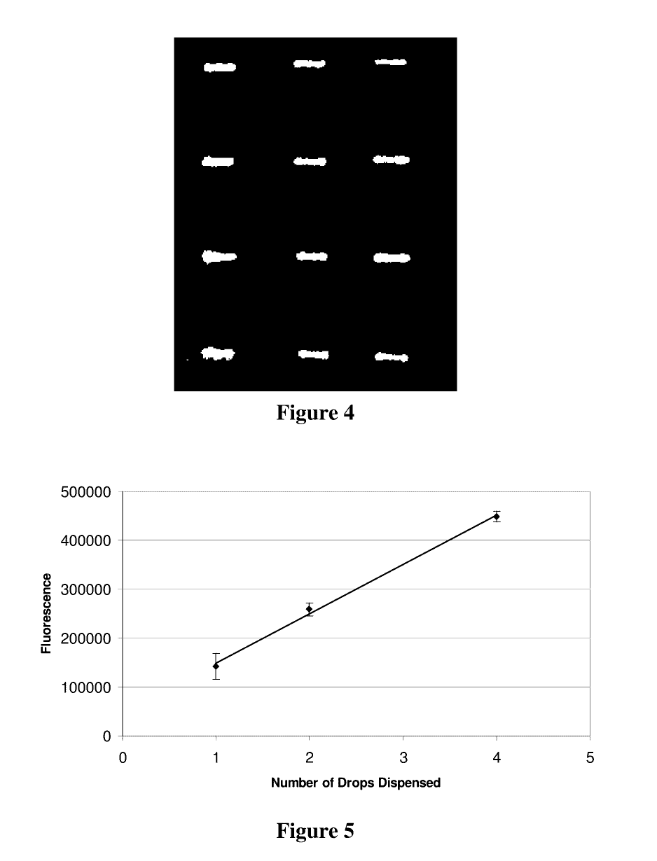

To assess volumetric linearity of dispensing, the dispensing pattern to a 96-well plate was set such 1 droplet was dispensed to each well along a first pair of 2 rows of 12 wells each (24 wells total), 2 droplets were dispensed to each well of the second pair of rows, and 4 droplets were dispensed to each well of the third pair of row. The averages and standard deviations are plotted against the number of droplets dispensed per well in FIG. 5. The points fall along a line having slope of 116,881 PMT counts per droplet, and the coefficient of determination for the fit is 0.9972.

Therefore, continuous liquid dispensing of the biochemical and biological assay compatible liquid formulations of the present invention are both volumetrically and spatially accurate and precise.

### Example 4

### Viability of Dispensed Cells

Murine neural stem cells (mNSC) were viable after dispensing as determined by their ability to proliferate and grow in culture. NSC were thawed from cryopreserved culture and grown in 6-well plates and T75 culture flasks. Cells were fed every other day with culture medium consisting of Dulbecco's Modified Essential Medium (DMEM) containing 4.5 grams per liter glucose, 5 mM sodium pyruvate, 5 mM GlutaMAX™ (L-alanyl-L-glutamine, Thermo Fisher Scientific, Carlsbad, Calif.), 10% (v:v) fetal bovine serum, 5% (v:v) horse serum, and 5 mM penicillin-streptomycin. Cells were incubated at 37° C. in an atmosphere containing 5% CO₂ and a relative humidity >95% for about 72 hrs until confluent. Prior to dispensing, cells were dissociated to singlets by brief incubation in trypsin-EDTA, collected by washing the culture work article with medium, and centrifuged at 1200 relative centrifugal force for 10 min. After aspiration of the supernatant, the remaining cell pellet was resuspended in 1.0 ml of medium. A 2 μl aliquot of this cell suspension was diluted into 1.0 ml PBS containing 0.2% Trypan Blue, briefly vortexed, and 10 μl transferred to a Neubauer hemocytometer for counting viable cells. This cell count was used to determine the volume of medium or PBS required to be added to the cell suspension to result in a final viable cell density of 10⁶ per milliliter. This density is equivalent to one cell per nanoliter, or an average of one cell per dispensed droplet. The cells were then placed in the external liquid reservoir of the SampleMaker with the end of the return fluid circuit above the liquid to avoid bubbling or foaming.

Cells in PBS or culture medium were dispensed using the SampleMaker to individual wells of 6-well culture plates using dispensing parameters identical to those used in Example 1. In each dry well, cells were dispensed as an 8×8 checkerboard pattern such that 32 droplets were dispensed at each actuation. This pattern was printed 2, 4, or 8 times in each well, such that on average, 64, 128, or 256 cells were dispensed to each well, respectively. Alternatively, cells were dispensed using the SoluDot banner logo pattern distributed across multiple wells of the plate. After the cells were dispensed to a plate, 5 ml culture medium was added to each well, and the plate was incubated at 37° C. for 3 days before observation.

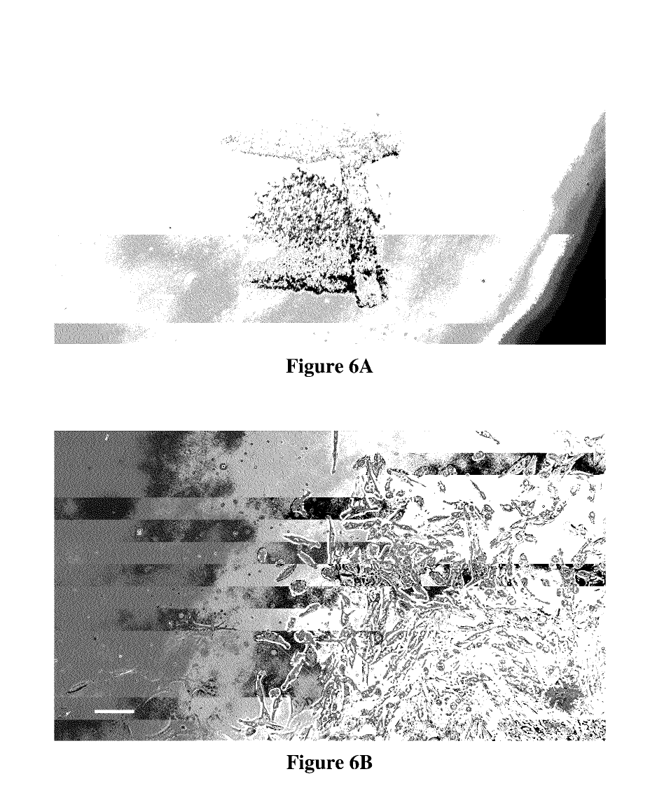

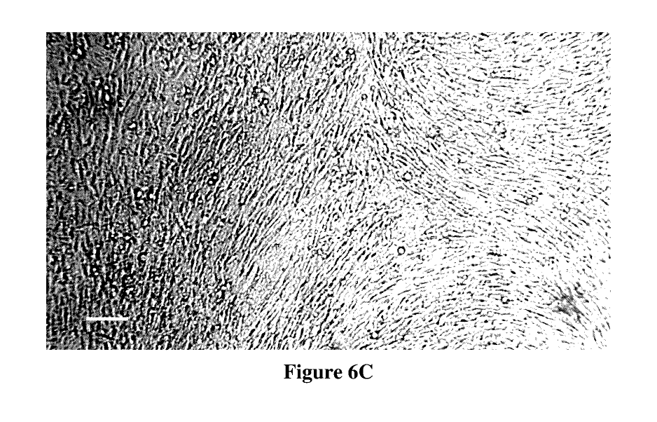

In FIGS. 6A-6C, a single actuation of the banner logo dispensing pattern was distributed across a row of 3 wells in a 6-well plate, FIG. 6A shows mNSC delivered to one well immediately after dispensing and filling the well with culture medium. These cells have not had an opportunity to grow, but their distribution resembles a portion of the dispensed logo pattern. FIG. 6B was obtained from the same well 3 days later. The right side of the image reveals abundant growth of the cells to cover part of the growth substrate, while the center of the image shows cell growth at the edge of the proliferating colony. These cells have adopted multipolar morphologies, show expression of cytosol that allows clear delineation between the cell nucleus and plasmalemma, and have extended lamellipodia and filopodia toward the cell-free left side of the image, characteristic of a motile phenotype. As mNSC undergo extensive migration during proliferation and growth, these dispensed cells are healthy. FIG. 6C shows cells in the same well 3 days after the acquisition of the image shown in FIG. 6B. By this time, 6 days after dispensing, the cells have grown to confluence, i.e., they cover the entire surface area of the growth substrate. This is further demonstration that at least some cells dispensed by our liquid-jetting method retain viability and are able to grow normally. Scale bars: FIG. 6A, 50 μm; FIGS. 6B and 6C, 10 μm.

1. A method for high-throughput dispensing of biocompatible liquid medium containing a biological sample which method comprises applying uniform droplets of said medium to predetermined positions on a target stage by dispensation by a continuous inkjet printer.

2. The method of claim 1 wherein the liquid medium is such that
the density is in the range of 900-1200 kg-m⁻³;
the surface tension is in the range of 7-10×10⁻² J-m⁻²; and
the viscosity is in the range of 7-15×10⁻⁴ Pa-s.

3. The method of claim 1 wherein said applying comprises generating droplets of uniform size from electrostrictive modulation of liquid passing from a dispensing reservoir through an orifice with said modulation having adjustable voltage frequency and amplitude applied to the electrostrictive element;
steering said droplets to predetermined positions on a target stage by charging the droplets through a pair of charging electrodes such that the amount of charge on each droplet determines the spatial position of said droplet on the target;
synchronizing the charge on each droplet with passage of the droplet between the charging electrodes by correction of the phase of application of the charging voltage with respect to the modulation signal; and
deflecting the droplets to said predetermined positions by passing said droplets through a deflection field.

4. The method of claim 1 wherein the biological sample comprises live cells and electrolyte compatible with live cells.

5. The method of claim 1 wherein the biological sample comprises nucleic acids and/or protein and electrolyte compatible with nucleic acid or protein.

6. The method of claim 3 wherein the amplitude of the modulation voltage is 15-27 volts; the net voltage difference of the deflection field is 5-7 kilo volts; and, when the orifice diameters are as set forth below, the frequency of the modulating voltage and pressure on the dispensing reservoir liquid are based on the relationships set forth as follows:

| | Modulating Frequency | |
|---|---|---|
| Orifice Diameter (μm) | KHz | Dispense pressure (mbar) |
| 36 | 105-110 | 3,400-3,700 |
| 42 | 100-105 | 2,700-3,200 |
| 55 | 90-95 | 2,400-2,700 |
| 70 | 65-70 | 2,300-2,500 |

7. A system for high-throughput dispensing of biocompatible medium containing a biological sample which system comprises a continuous inkjet printer and at least one computerized control unit wherein said control unit(s) adjusts the operating parameters of the continuous inkjet printer to be satisfactory for dispensing said medium.

8. The system of claim 7 wherein said liquid medium is such that
the density is in the range of 900-1200 kg-m⁻³;
the surface tension is in the range of 7-10×10⁻² J-m⁻²; and
the viscosity is in the range of 7-15×10⁻⁴ Pa-s.

9. The system of claim 7 wherein the inkjet printer comprises a dispensing reservoir, an orifice through which said medium is dispensed, modulating element for vesiculating said liquid into droplets, charging electrodes to provide electrical charge to said droplets, an electric field for deflection of said droplets, and
wherein said control unit(s) adjusts the pressure in the liquid dispensing reservoir, the frequency of the modulating electrodes, the voltage difference between the charging electrodes and the net voltage difference of the deflecting electric field.

10. The system of claim 9 wherein the amplitude of the modulation voltage is 15-27 volts; the net voltage difference of the deflection field is 5-7 kilo volts; and, when the orifice diameters are as set forth below, the frequency of the modulating voltage and pressure on the dispensing reservoir liquid are based on the relationships set forth below:

| | Modulating Frequency | |
|---|---|---|
| Orifice Diameter (μm) | KHz | Dispense pressure (mbar) |
| 36 | 105-110 | 3,400-3,700 |
| 42 | 100-105 | 2,700-3,200 |
| 55 | 90-95 | 2,400-2,700 |
| 70 | 65-70 | 2,300-2,500 |
